(19) United States
(12) Patent Application Publication
(10) Pub. No.: US 2026/0232952 A1
(43) Pub. Date: Aug. 13, 2026

(54) METHOD FOR PRODUCING A CATHETER BODY

(71) Applicants: Heraeus Medevio GmbH & Co. KG, Hanau (DE); Contract Medical International GmbH, Dresden (DE)

(72) Inventors: Ilias NIKOLAIDIS, Hanau (DE); Mario HEINTZE, Dresden (DE); Llewellyn Wallace GROENEVELD, Dresden (DE); Raffael KUNZ, Dresden (DE)

(21) Appl. No.: 19/531,127

(22) Filed: Feb. 5, 2026

(30) Foreign Application Priority Data
Feb. 10, 2025 (DE) 102025104696.4

(51) Int. Cl. A61M 25/00; A61B 17/00; A61B 18/00

(52) U.S. Cl. CPC A61M 25/0012; A61B 2017/00526; A61B 18/00; A61B 2018/00077; A61B 2018/00083; A61B 2018/00178; A61M 2207/00

(57) ABSTRACT

A method for producing a catheter body, including the steps of a) providing a hollow-cylindrical inner liner having an inner lumen and a radially outer inner liner outer surface, wherein the inner liner comprises a thermoset; b) creating a plurality of anchoring recesses in the inner liner which extend from the inner liner outer surface towards the inner lumen; c) providing a flexible circuit board comprising a plurality of electrical conductors; d) arranging the flexible circuit board on the inner liner outer surface; e) sheathing the inner liner and the flexible circuit board with an outer insulation, wherein the outer insulation comprises a thermoplastic and is at least partially introduced into the anchoring recesses; f) providing at least one electrode; and, g) establishing electrical contact between the at least one electrode and at least one of the electrical conductors of the flexible circuit board.

### CROSS-REFERENCE TO RELATED APPLICATION

This application claims priority pursuant to 35 U.S.C. 119(a) to German Patent Application No. 102025104696.4, filed February 10, 2025, which application is incorporated herein by reference in its entirety.

### FIELD OF THE INVENTION

The invention relates to a method for producing a catheter body, in particular for an ablation catheter or a mapping catheter, comprising the method steps of:
a) providing a hollow-cylindrical inner liner having an inner lumen and a radially outer inner liner outer surface, wherein the inner liner comprises a thermoset;
b) creating a plurality of anchoring recesses in the inner liner which extend from the inner liner outer surface towards the inner lumen;
c) providing a flexible circuit board comprising a plurality of electrical conductors;
d) arranging the flexible circuit board on the inner liner outer surface;
e) sheathing the inner liner and the flexible circuit board with an outer insulation, wherein the outer insulation comprises a thermoplastic and is at least partially introduced into the anchoring recesses;
f) providing at least one electrode;
g) establishing electrical contact between the at least one electrode and at least one of the electrical conductors of the flexible circuit board.

### BACKGROUND

Catheters have been used in medical procedures for many years. Among other things, catheters can be used in medical procedures to examine, diagnose, and/or treat tissue in particular in areas of the body, such as blood vessels, that are difficult to access without more invasive procedures. For example, catheters can be used to deliver electrical energy to selected locations in the human body, like the heart, and thereby kill tissue, like heart tissue. This is often referred to as “ablation.” Catheters can also be used to stimulate body tissue. During stimulation, the amount of energy transferred to the body tissue is usually lower than during ablation.

Another procedure, often called "mapping," uses a catheter with sensing electrodes to monitor various forms of bioelectrical activity in the human body.

Catheters can therefore be used both to transmit (during “ablation” and “stimulation”) and to absorb (during “mapping”) energy to or from body tissue.

Regardless of the direction of energy flow, catheters often contain a plurality of electrodes, i.e., two or more, at a distal catheter body end region, wherein the distal catheter body end region often describes the end that is inserted into the patient. Typically, the catheter contains an equal number of electrodes at its proximal catheter body end region, which are electrically connected to the electrodes at the distal catheter body end region. The electrodes at the proximal catheter body end region are used to connect electrical devices according to the desired areas of application of the catheter.

In principle, attempts are made to keep the outer diameter of a catheter, in particular for ablation catheters and mapping catheters, as small as possible in order to minimize the risk of damage to the blood vessels of the patient during their introduction and passage. However, this encounters some hurdles in practice. On the one hand, the electrodes, in particular ring electrodes, are usually connected electrically with wires or cables that run in an inner lumen of the catheter body, depending upon their intended use. These take up a significant portion of the diameter of the inner lumen which, depending upon the intended further use of the inner lumen, e.g., for a guide wire, makes a larger inner lumen necessary and, with constant wall thicknesses, therefore also a larger outer diameter of the catheter body. Furthermore, due to their ease of processing, thermoplastics, i.e., thermoplastic polymers, are generally used to produce the catheter bodies. However, these have reduced mechanical stability in comparison to other polymers, which is why the walls of the catheter bodies made of thermoplastics are usually equipped with greater wall thicknesses and often additionally with reinforcing structures, such as embedded metal coils or metal braids, for mechanical reinforcement. Both lead to an enlarged outer diameter of the catheter body with an unchanging inner lumen.

Thermosets, which have increased mechanical stability in comparison to thermoplastics, are more involved to process and, in particular with greater wall thicknesses, have less flexibility, which can limit their use as catheter body material. A combination of thermoplastics and thermosets for catheter bodies usually fails, since the two material classes bond poorly.

There is therefore a need in the market for an improved production method for catheter bodies, in particular for catheter bodies for ablation catheters and mapping catheters, which allows the production of catheter bodies that are as mechanically stable as possible while at the same time having the smallest possible wall thicknesses.

### SUMMARY

One object of the present invention is to overcome, at least in part, one or more of the disadvantages resulting from the prior art.

In particular, it is an object of the present invention to provide a method for producing a catheter body, in particular a catheter body for an ablation catheter or a mapping catheter, which allows the production of such a catheter body in a simple, safe, cost-effective, and flexible manner. The method shall allow the production of a catheter body with the smallest possible outer diameter. Furthermore, the method shall allow the production of catheter bodies that are as mechanically stable as possible, while at the same time having the smallest possible wall thickness. The method is further intended to provide a catheter body which leaves as much of the inner lumen of the catheter body as possible freely available during its use.

### PREFERRED EMBODIMENTS OF THE INVENTION

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a catheter body, comprising the method steps of:
a) providing a hollow-cylindrical inner liner having an inner lumen and a radially outer inner liner outer surface, wherein the inner liner comprises a thermoset;
b) creating a plurality of anchoring recesses in the inner liner which extend from the inner liner outer surface towards the inner lumen;
c) providing a flexible circuit board comprising a plurality of electrical conductors;
d) arranging the flexible circuit board on the inner liner outer surface;
e) sheathing the inner liner and the flexible circuit board with an outer insulation, wherein the outer insulation comprises a thermoplastic and is at least partially introduced into the anchoring recesses;
f) providing at least one electrode; and
g) establishing electrical contact between the at least one electrode and at least one of the electrical conductors of the flexible circuit board.

In a preferred embodiment of the method, the anchoring depressions have a diameter in a range between 5 µm and 200 µm, preferably in a range between 10 µm and 100 µm, more preferably in a range between 20 µm and 50 µm. This embodiment is a second embodiment of the invention, which is preferably dependent upon the first embodiment of the invention.

In a preferred embodiment of the method, the anchoring recesses have a radial depth which is greater than their diameter. This embodiment is a third embodiment of the invention, which is preferably dependent upon the second embodiment of the invention.

In a preferred embodiment of the method, the anchoring recesses extend from the inner liner outer surface into the inner lumen. This embodiment is a fourth embodiment of the invention, which is preferably dependent upon one of the preceding embodiments of the invention.

In a preferred embodiment of the method, in method step e), the outer insulation is introduced, via the anchoring recesses, into the inner lumen. This embodiment is a fifth embodiment of the invention, which is preferably dependent upon the fourth embodiment of the invention.

In a preferred embodiment of the method, the sheathing in method step e) comprises extruding the outer insulation. This embodiment is a sixth embodiment of the invention, which is preferably dependent upon one of the preceding embodiments of the invention.

In a preferred embodiment of the method, the sheathing in method step e) comprises exerting pressure on the outer insulation in the direction of the inner lumen. This embodiment is a seventh embodiment of the invention, which is preferably dependent upon one of the preceding embodiments of the invention.

In a preferred embodiment of the method, the pressure is exerted on the outer insulation by means of a heat-shrink tube. This embodiment is an eighth embodiment of the invention, which is preferably dependent upon the seventh embodiment of the invention.

In a preferred embodiment of the method, the outer insulation is formed by heating a heat-shrink tube. This embodiment is a ninth embodiment of the method, which is preferably dependent upon the seventh embodiment of the invention.

In a preferred embodiment of the method, the creation of the anchoring recesses in method step b) comprises drilling, punching, cutting, or a laser processing method such as laser ablation. This embodiment is a tenth embodiment of the invention, which is preferably dependent upon one of the preceding embodiments of the invention.

In a preferred embodiment of the method, the sheathing in method step e) comprises heating the outer insulation. This embodiment is an eleventh embodiment of the invention, which is preferably dependent upon one of the preceding embodiments of the invention.

In a preferred embodiment of the method, the inner liner provided in method step a) has a wall thickness in a range between 10 µm and 200 µm, preferably in a range between 20 µm and 100 µm, more preferably in a range between 25 µm and 75 µm. This embodiment is a twelfth embodiment of the invention, which preferably depends upon one of the preceding embodiments of the invention.

In a preferred embodiment of the method, at least one contact opening is created in the outer insulation for establishing electrical contact with the electrode in method step g) in order to selectively establish electrical contact between the electrode and at least one electrical conductor, accessible via the contact opening, of the flexible circuit board. This embodiment is a thirteenth embodiment of the invention, which is preferably dependent upon one of the preceding embodiments of the invention.

In a preferred embodiment of the method, the arrangement in method step d) comprises a spiral winding of the flexible circuit board in windings around the inner liner, and the creation of the anchoring recesses in method step b) takes place in such a way that, in a plan view of the catheter body perpendicular to its longitudinal axis, i.e., in a lateral view of the catheter body, at least one anchoring recess is positioned between every two axially adjacent windings. This embodiment is a fourteenth embodiment of the invention, which preferably depends upon one of the preceding embodiments of the invention.

In a preferred embodiment of the method, the inner liner provided in method step a) comprises a polyimide (PI), a polyether ether ketone (PEEK), or consists of a polyimide or a polyether ether ketone. This embodiment is a fifteenth embodiment of the invention, which is preferably dependent upon one of the preceding embodiments of the invention.

### IN GENERAL

In addition to the embodiments described herein, the elements of which “contain” or “comprise” a particular feature (e.g., a material), a further embodiment is always contemplated in which the element in question consists solely of the feature, i.e., does not comprise any other components. The word “comprise” or “comprising” is herein used synonymously with the word “contain” or “containing.”

When, in an embodiment, an element is referred to in the singular, an embodiment is also contemplated in which several of these elements are present. The use of a term for an element in the plural generally also includes an embodiment in which only a single corresponding element is included. Unless otherwise stated or, from the context, clearly excluded, it is fundamentally possible and is hereby clearly considered that features of different embodiments can also be provided in the other embodiments described herein. It is also generally contemplated that all features described herein in connection with a method are also applicable to the products and devices described herein, and vice versa. Merely for the sake of conciseness, these considered combinations are not all explicitly listed in all cases. Technical solutions that are known to be equivalent to the features described herein shall also be included in principle in the scope of the invention.

In the present description, range specifications also include the values specified as limits. An indication of the type “in the range of X to Y” with respect to a size A consequently means that A can assume the values X, Y and values between X and Y. Ranges which are limited on one side, of the type "up to Y" for a size A, accordingly mean a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter,” or “parallelism” can never be given exactly, but only within certain manufacturing error tolerances. For example, "substantially perpendicular axes" form an angle of 85 degrees to 95 degrees relative to one another, and "substantially equal volumes" comprise a variation of up to 5 vol.%. For example, a "device consisting substantially of plastic" comprises a plastic content of ≥95 to ≤100 wt.%. For example, a "substantially complete filling of a volume B" comprises a filling of ≥95 to ≤100 vol.% of the total volume of B.

A first subject matter of the invention relates to a method for producing a catheter body comprising the following method steps: a) providing a hollow-cylindrical inner liner having an inner lumen and a radially outer inner liner outer surface, wherein the inner liner comprises a thermoset; b) creating a plurality of anchoring recesses in the inner liner which extend from the inner liner outer surface towards the inner lumen; c) providing a flexible circuit board comprising a plurality of electrical conductors; d) arranging the flexible circuit board on the inner liner outer surface; e) sheathing the inner liner and the flexible circuit board with an outer insulation, wherein the outer insulation comprises a thermoplastic and is at least partially introduced into the anchoring recesses; f) providing at least one electrode; and, g) establishing electrical contact between the at least one electrode and at least one of the electrical conductors of the flexible circuit board.

The method serves to produce a catheter body for a catheter, in particular for an ablation catheter or a mapping catheter. A catheter body is the elongated, tube-like portion of a catheter which, depending upon the application of the catheter, is equipped with a plurality of electrodes, at least at a distal catheter body end, preferably both at the distal and at a proximal catheter body end axially opposite the distal catheter body end. The electrodes at the proximal end of the catheter body are sometimes called connectors.

In a method step a), a hollow-cylindrical inner liner with a radially outer inner liner outer surface is provided. The inner liner extends axially from a proximal catheter body end to a distal catheter body end and radially surrounds at least one inner lumen extending axially through the inner lining and therefore also the final catheter body.

The inner lumen, or at least a part thereof, can, for example, serve to pass through and/or introduce various devices. For example, the inner lumen can serve to insert a guide wire, which facilitates the introduction of the catheter into the patient. Furthermore, the inner lumen, or a part thereof, can serve to pass a fluid, such as a cooling liquid, which can be discharged from the catheter at a suitable point in order to cool the surrounding body tissue, for example. If the inner lumen serves multiple purposes, e.g., the passage of a cooling liquid and the introduction of a guide wire, it can be divided into several compartments by means of inner wall(s) in order to create separate sections - for example, for the cooling liquid and the guide wire. Such catheters with a divided inner lumen are often also referred to as multi-lumen catheters.

The inner liner comprises a thermoset, or in other words a thermosetting polymer. Thermosets are sometimes also called duromers. Thermosets are polymers which, after they cure, which often occur through heating, exhibit increased mechanical strength, in particular in comparison to thermoplastics, due to their often tightly meshed cross-linking.

The inner liner can comprise a mixture of different thermosets, wherein it is preferred that the inner liner consist substantially of a single thermoset and, optionally, minor amounts of additives, such as dyes.

An inner liner comprising a thermoset or, preferably, consisting of a thermoset, enables a smaller wall thickness of the inner liner in comparison to an inner liner comprising a thermoplastic, as is usually the case with commercial catheter bodies. This results in a smaller outer diameter of the catheter body for the same inner lumen diameter of the inner liner, or, for the same outer diameter of the catheter body, in an enlarged inner lumen.

In order to ensure a sufficiently high flexibility of the inner liner despite the increased mechanical stability in comparison to thermoplastics, it is preferred that the inner liner have a wall thickness in a range between 10 µm and 200 µm, preferably in a range between 20 µm and 100 µm, more preferably in a range between 25 µm and 75 µm. This range represents a good compromise between flexibility and mechanical stability of the inner liner.

The inner liner has an inner liner inner diameter which lies, for example, in a range of 0.75 to 4 mm. Depending upon the application, the inner liner can have different lengths. For example, the inner liner has a length of 30 cm to 200 cm. The inner liner inner diameter preferably corresponds to the diameter of the inner lumen.

The provision of the inner liner can be accomplished in different ways. In one embodiment, the provision comprises coating a mandrel, e.g., made of copper, with a precursor or monomer of a thermoset, wherein the layer on the mandrel is cured - for example, by heating. The coating can be achieved, for example, by immersion in the precursor or the monomer. After removing the mandrel and, optionally, removing unwanted sections, the inner liner is ready. In particular, the preferred low wall thicknesses of the inner liner can be realized easily and economically by means of such a coating.

In a method step b), a plurality of, i.e., two or more, anchoring recesses are created in the inner liner, which extend from the outer surface of the inner liner in the direction of the inner lumen. The anchoring recesses serve to improve the adhesion of the outer insulation (see method step e), which comprises a thermoplastic and therefore would not provide sufficient adhesion to the inner liner on its own. Without the anchoring recesses, there would be a risk of spatial separation of the inner liner and the outer insulation, in particular when inserting the catheter body into a blood vessels of a patient. The anchoring recesses therefore increase the mechanical stability of the catheter body.

The anchoring recesses represent tunnel-like notches in the surface of the inner liner, which extend radially inwards, i.e., in the direction of the inner lumen, wherein these tunnel-like notches are at least partially filled with the outer insulation during the method (see method step e), and the outer insulation is therefore arranged with improved mechanical adhesion to the inner liner.

In a method step c), a flexible circuit board comprising a plurality of electrical conductors is provided. The flexible circuit board, sometimes also called flex circuit ribbon, is a circuit board comprising or consisting of flexible, bendable materials that allow the circuit board to be bent or folded or wrapped into different shapes. The structure of a typical flexible circuit board comprises several layers. The core element is one or more electrically conductive layer(s), e.g., made of a metal foil or a printed metal layer preferably comprising copper or consisting of copper, which can contain a specific pattern, depending upon the type of application. This layer or a conductor track within this layer acts as the electrical conductor of the flexible circuit board. The electrically conductive layers are usually embedded between electrically insulating layers made of flexible insulation material such as polyimides, which can also provide mechanical protection.

The flexible circuit board of the method according to the invention comprises a plurality of electrical conductors, i.e., at least two. For example, the flexible circuit board comprises two to 20 electrical conductors, each of which can be electrically connected to one or more electrodes. For example, the flexible circuit board comprises 12 electrical conductors which can be selectively connected to 12 electrodes of the catheter body. Preferably, the electrical conductors are electrically isolated from each other. More preferably, the flexible circuit board comprises at least two insulating layers which electrically insulate the at least one electrical conductor from the outside at least over long sections of the flexible circuit board. The flexible circuit board can have contact surfaces at certain positions which make the electrical conductor electrically contactable from the outside without the need to remove part of the insulating layer beforehand.

An advantage of using a flexible circuit board is its small space requirement, low weight, and simplified installation. In particular, the use of a flexible circuit board eliminates the need for installing individual conductive wires or cables for electrically contacting the electrodes of the catheter body, particularly through the inner lumen of the inner liner.

In a method step d), the flexible circuit board is arranged on the inner liner outer surface. The flexible circuit board is preferably arranged in such a way that the flexible circuit board allows an electrical connection of electrodes at the distal catheter body end of the catheter body with electrodes at the proximal catheter body end. After being arranged, the flexible circuit board therefore extends along additional parts of the inner liner, e.g., along at least 70%, preferably along at least 80%, more preferably along at least 90%, of a total length of the inner liner. In one embodiment, the flexible circuit board extends substantially over the entire length of the inner liner. Arranging the flexible circuit board on the inner liner outer surface can comprise simply placing it on the inner liner such that the longitudinal axes of the inner liner and the flexible circuit board are aligned substantially parallel to each other, for example. In order to simplify the arrangement and the subsequent method steps, in particular method step d), it can be advantageous to fix the flexible circuit board to the inner liner outer surface, at least temporarily, e.g., by means of an adhesive such as an adhesive or an adhesive tape - for example, by means of a double-sided adhesive tape.

In a method step e), the inner liner and the flexible circuit board arranged on the inner liner outer surface are sheathed, in particular coaxially sheathed, or at least partially sheathed, with an outer insulation comprising a thermoplastic. The sheathing is carried out in such a way that the outer insulation at least partially fills the anchoring recesses. Preferably, the majority of the outer insulation remains outside the anchoring recess and radially outwards with respect to the inner liner outer surface, and a relatively smaller portion is introduced into the anchoring recesses. The anchoring recesses can be filled completely or only partially with the material of the outer insulation. The part of the outer insulation introduced into the anchoring recesses improves the adhesion of the outer insulation to the inner liner, which would be significantly less mechanically stable without anchoring recesses due to the different material properties of thermosets and thermoplastics. In particular, the anchoring recesses at least partially filled with the outer insulation reduce the risk of displacement of the outer insulation relative to the inner liner, which would happen in particular when inserting and passing the catheter body through blood vessels with the material combination of thermoset and thermoplastic.

By sheathing the inner liner, it is coaxially surrounded by the outer insulation, wherein the inner liner outer surface faces an insulation inner surface. The outer insulation is preferably the radially outer layer of the catheter body and serves in particular to electrically insulate the catheter body from the outside and to mechanically protect the catheter body. Preferably, the outer insulation has an outer diameter in a range of 200 µm to 5,000 µm, more preferably 300 µm to 3,000 µm and most preferably 500 µm to 1,500 µm. In an exemplary embodiment, the outer diameter of the outer insulation is about 700 µm. It is understood that the outer diameter of the outer insulation can also correspond to the outer diameter of the catheter body. The outer insulation preferably has a wall thickness in the range of 2 µm to 300 µm, particularly preferably 5 µm to 150 µm, and most preferably 20 µm to 100 µm. According to a preferred embodiment of the present invention, the outer insulation has an outer diameter in a range of 300 µm to 3,000 µm, particularly preferably 500 µm to 1,500 µm, and a wall thickness in a range of 5 µm to 150 µm, particularly preferably 20 µm to 100 µm. In an exemplary embodiment, the wall thickness is approximately 60 µm. The outer insulation preferably consists of a thermoplastic polymer that is selected from the group of silicones, polyolefins (e.g., polyethylene), polyether block amide (e.g., PEBAX®), polyurethanes, polyamides, polyarylether ketones (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof. According to a preferred embodiment, the outer insulation comprises a polymer that is selected from the group of silicones, polyolefins (e.g., polyethylene), polyurethanes, polyamides, polyarylether ketone (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof, and has an outer diameter in the range of 300 to 3,000 µm.

The flexible circuit board is arranged radially between the inner liner and the outer insulation, i.e., between the inner insulation surface and the outer inner liner surface.

An advantage of an external insulation comprising a thermoplastic, preferably consisting of a thermoplastic or a thermoplastic mixture, is that it can be produced easily and inexpensively. Furthermore, thermoplastics are more flexible in comparison to thermosets, so that the final catheter body has sufficient flexibility, which would not be possible or only slightly possible with a catheter body exclusively of thermosets, and if so, only with very small wall thicknesses. Insufficient wall thickness of the catheter body carries the risk of kinking and breaking.

Furthermore, the use of a thermoplastic can enable the flexible circuit board to be fixed between the inner liner and the outer insulation, which is preferred. This fixing of the flexible circuit board preferably substantially prevents free movement of the flexible circuit board relative to the inner liner and/or the outer insulation. The location and orientation, or in other words the position, of the flexible circuit board within the catheter body is therefore fixed and basically no longer changes due to the creation of the connection between the inner liner and the outer insulation, which was created by at least partial penetration of the outer insulation into the anchoring recesses of the inner liner, which represents an advantage in comparison to the prior art over the wires or cables normally arranged in the inner lumen for electrically contacting the electrodes of the catheter body. The fixed flexible circuit board, whose position and orientation are either known or can be easily determined, e.g., by using a transparent outer insulation or by applying markings to the outer surface of the catheter body, can significantly simplify the further method steps. Depending upon the design of the method, the position and orientation of the flexible circuit board can also be stored electronically - for example, in a CAT file. For example, the flexible circuit board fixed in this way allows for the subsequent creation of flushing feedthroughs without the risk of unwanted severing of the electrical conductors of the flexible circuit board. Furthermore, fixing the flexible circuit board reduces the risk of bending or breaking the flexible circuit board, in particular its electrical conductors.

In a method step f), at least one electrode is provided. The exact number of electrodes depends upon the how the catheter body is used. Preferably, as many electrodes as electrical conductors are provided, so that each of the electrodes can be electrically connected to an electrical conductor during the method.

Preferably, the electrodes are ring electrodes. Further preferably, all provided electrodes are ring electrodes.

The electrode can comprise a plurality of different materials or consist of different materials. Preferably, the electrode comprises a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode - for example, stainless steel AISI 316L, stainless steel AISI 301, or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) can depend upon the use of the catheter according to the invention. However, it should be understood that the application of the catheter is not limited by the use of a particular metal.

The electrode can also have a coating. Suitable coatings are metal nitrides such as TiN, metal oxides such as IrO₂, or conductive polymers. The surface of the electrode can also be surface-structured, e.g., laser-structured.

The electrode, preferably a ring electrode, can have an outer diameter in the range of 200 to 5,000 µm, in one embodiment in the range of 300 to 3,000 µm, and in one embodiment in the range of 500 to 1,500 µm. The electrode can have a wall thickness in the range of 10 to 200 µm, in one embodiment 10 to 100 µm, and in one embodiment 30 to 70 µm. Furthermore, the electrode can have a length in the range of 200 to 5,000 µm, in one embodiment 300 to 3,000 µm, and in one embodiment in the range of 500 to 1,500 µm. According to one embodiment, the electrode has an outer diameter in the range of 300 to 3,000 µm and in one embodiment in the range of 500 to 1,500 µm, a wall thickness in the range of 10 to 100 µm and in one embodiment of 30 to 70 µm, and a length in the range of 300 to 3,000 µm and in one embodiment in the range of 500 to 1,500 µm.

In a method step g), the at least one electrode, preferably all electrodes, are in electrical contact with at least one of the electrical conductors, preferably all electrodes with one, preferably with exactly one, electrical conductor of the flexible circuit board. Depending upon the design, establishing the electrical contact can include removing parts of the insulation of the flexible circuit board so that the corresponding electrical conductor(s) are available for establishing electrical contact with the electrode. Establishing the electrical contact can also include a mechanical fixation of the electrode to the catheter body. In one embodiment, the at least one electrode is brought into direct physical contact with the at least one electrical conductor in order to establish the electrical contact. In a further embodiment, the at least one electrode and the at least one electrical conductor are not electrically connected to one another via direct physical contact, but, rather, indirectly via an electrically conductive bridge element. Examples of bridge elements include metal elements such as metal plates, metal spring elements, or metal balls, as well as conductive adhesives, conductive pastes, or solders.

The method steps a) to g) of the process according to the invention can, if technically possible and reasonable, be carried out in different sequences and/or at least partly simultaneously.

For example, method steps a) and b) can take place simultaneously. In this embodiment of the method, the anchoring recesses are created directly during the production of the inner liner. For example, this can be done by immersing a mandrel with protruding elevations on the surface of the mandrel into a precursor or monomer of the thermoset. The elevations form the anchoring depressions during the hardening of the initial mixture. After curing of the precursor or monomers and removal of the mandrel, the inner liner including the anchoring recesses are ready.

A preferred embodiment of the process is characterized in that method step e) is carried out before method steps f) and g). In this embodiment, the inner liner and the flexible circuit board are sheathed with the outer insulation before the installation of the at least one electrode. Preferably, the sheathing fixes the flexible circuit board between the inner liner and the outer insulation so that it can basically no longer move freely relative to the catheter body. Since the position of the electrical conductors is known by fixing the flexible circuit board, e.g., electronically recorded, e.g., by means of a CAT file, or easily experienced, e.g., by using a transparent outer insulation, and also basically no longer changes, this sequence can simplify establishing the electrical contact with the at least one electrode. Furthermore, this sequence increases the flexibility of the method, since an otherwise identical catheter body can be easily manufactured in different positions with electrodes and with a different number of electrodes.

In one embodiment, method steps e) and g) take place simultaneously. In this embodiment, it is preferred that the sheathing of the inner liner in method step d) comprise a partial sheathing of the inner liner and the flexible circuit board, which is carried out by sliding on a hollow-cylindrical outer insulation. Before method step e) is carried out, the at least one electrode, preferably in the form of a ring electrode, is pushed onto the inner liner to the desired position and orientation relative to the flexible circuit board. This process can be repeated with several outer insulations and electrodes, which are preferably pushed alternatingly onto the inner liner. Subsequently, method steps e) and g) take place simultaneously, preferably by heating. Furthermore, it can be advantageous to realize the electrical contact with the at least one electrode via an electrically conductive bridge element - for example, in the form of a solder paste or a conductive adhesive.

The anchoring recesses can have different dimensions, in particular diameters and depths, in order to create a sufficient connection of the outer insulation to the inner liner. In particular, the dimension of the anchoring recesses can be selected depending upon the choice of employed materials, in particular the employed thermoplastic or thermoplastic mixture for the external insulation and the way in which this material is introduced into the anchoring recesses. Furthermore, the dimension of the anchoring recesses can be selected depending upon the dimensions, in particular the wall thicknesses, of the components of the catheter body, in particular the dimension of the outer insulation and in particular the inner liner.

A preferred embodiment of the method is characterized in that the anchoring depressions have a diameter in a range between 5 µm and 200 µm, preferably between 10 µm and 100 µm, more preferably between 20 µm and 50 µm. This range normally allows sufficient penetration of the outer insulation into the anchoring recesses to achieve a good connection to the inner liner and does not have too negative an impact on the mechanical stability of the inner liner. The diameter of an anchoring recess is the shortest distance between two opposite points on the opening, i.e., adjacent to the inner liner outer surface, of the anchoring recess. For round anchoring recesses, this is the length of a distance that runs through the center of the opening and touches both edges of the anchoring recess. For non-circular anchoring recesses, the diameter is the shortest distance that connects two opposite points on the opening. The diameter refers to the diameter of the opening of the anchoring recess, which can widen or narrow with increasing radial depth of the anchoring recess.

In addition to the diameter of the anchoring recess, its radial depth, i.e., the distance that extends from the opening towards the inner lumen, is also important. The radial depth of the anchoring recess is limited by the wall thickness of the inner liner at the corresponding position.

A preferred embodiment of the method is characterized in that the anchoring recesses have a radial depth that is greater than their diameter. In this embodiment, the anchoring recesses are “deeper” than they are “wide.” This can lead to improved adhesion of the outer insulation to the inner liner, since the part of the outer insulation located in the anchoring recesses is less easy to remove from the anchoring recess, e.g., by bending, if the penetration depth into the anchoring recesses is correspondingly deep.

As already mentioned, the radial depth of the anchoring recesses is limited by the wall thickness of the inner liner. For example, the anchoring recesses can have a radial thickness which corresponds to at least 30%, preferably at least 50%, more preferably at least 70% of the wall thickness of the inner liner. For example, the anchoring recesses can have a radial depth which corresponds to a range between 30% and 90%, preferably a range between 50% and 90%, more preferably a range between 70% and 90%, of the wall thickness of the inner liner at the position of the respective anchoring recess.

A preferred embodiment of the method is characterized in that the anchoring recesses extend from the inner liner outer surface into the inner lumen. In this embodiment, the anchoring recesses have a radial depth that corresponds to the wall thickness of the inner liner at the corresponding position. The anchoring recesses therefore constitute a kind of passage through the inner liner.

Anchoring recesses that extend from the inner liner outer surface into the inner lumen can facilitate the introduction of the outer insulation into them, since the gas present in the anchoring recess before introduction can be displaced into the inner lumen. In particular, basically, complete filling of the anchoring recesses is therefore made easier.

A preferred embodiment of the method is characterized in that, in method step e), the outer insulation is introduced via the anchoring recesses into the inner lumen. This means that part of the outer insulation extends into the inner lumen. Preferably, the inner lumen is not filled with the outer insulation in such a way that the diameter of the inner lumen is thereby significantly changed, in particular not significantly reduced. The outer insulation preferably extends only up to 0.5 mm into the inner lumen. Furthermore, it is preferred that the outer insulation be introduced into the inner lumen only far enough that its thermoplastic spreads slightly, e.g., up to 1 mm, over the inside of the inner liner, to improve the adhesion of the outer insulation to the inner liner. With a basically symmetrical spread over the inside of the inner liner, adhesion can be improved by a kind of undercut effect.

The method step e) can be carried out in different ways.

A preferred embodiment of the method is characterized in that the sheathing in method step e) comprises extrusion, in particular coaxial extrusion, of the outer insulation onto or around the inner liner. Extrusion is advantageous due to its simplicity and cost-effectiveness.

By coaxial extrusion, it can be ensured that the outer insulation is applied evenly and precisely to the inner liner. This results in a homogeneous layer that reliably sheathes the flexible circuit board and the inner liner. In addition, the viscous state of the thermoplastic during extrusion can allow the outer insulation to penetrate deeply into the anchoring recesses. This improves the mechanical adhesion and stability of the outer insulation, since it is firmly anchored in the anchoring recesses.

Another advantage of extrusion is the ability to precisely control the wall thickness of the outer insulation. This is particularly important for keeping the outer diameter of the catheter body to a minimum and at the same time for ensuring adequate insulation and mechanical strength. By using thermoplastics that are converted into a viscous state during extrusion, the outer insulation can be efficiently introduced into the anchoring recesses without additional processing steps, which reduces the overall cost and complexity of the production method.

The introduction of the external insulation into the anchoring recesses can be carried out in different ways. For example, the outer insulation can be heated so that the thermoplastic or the mixture of thermoplastics softens, and the softened material is basically introduced into the anchoring recesses independently - for example, by utilizing capillary action and/or gravity.

A preferred embodiment of the method is characterized in that the sheathing in method step e) comprises exerting a pressure on the outer insulation in the direction of the inner lumen. The pressure that is exerted from radially outside to radially inside facilitates, or, depending upon the material properties and material consistency, even enables, the introduction into the anchoring recesses.

The radially inward pressure can be exerted in different ways. For example, an atmospheric overpressure acts from outside upon the outer insulation so that it is introduced into the anchoring recesses.

A preferred embodiment of the method is characterized in that the pressure is exerted on the outer insulation by means of a heat-shrink tube. A heat-shrink tube is a hollow-cylindrical tube that is made of a material, often a polyolefin, polyvinyl chloride or fluoropolymer, which contracts radially to a fraction of its original diameter when heated to a certain temperature. This contraction can exert a pressure exerted radially inwards on the outer insulation. To do this, the heat-shrink tube is applied around the outer insulation, already arranged around the inner liner and the flexible circuit board, so that it is introduced into the anchoring recesses. The heating of the heat-shrink tube is preferably designed in such a way that the outer insulation softens so that introduction into the anchoring recesses is facilitated. After the outer insulation has been introduced into the anchoring recesses, the shrunken heat-shrink tube can be removed, either directly or after further method steps. Preferably, the heating also at least softens the outer insulation so that introduction into the anchoring recesses is facilitated.

A preferred embodiment is characterized in that the outer insulation is formed by heating a heat-shrink tube. In this embodiment, a shrunken heat shrink tube forms the outer insulation. When the heat-shrink tube is radially contracted, part of the material, preferably a polyolefin, polyvinyl chloride, or fluoropolymer, is at least partially introduced into the anchoring recesses.

The anchoring recesses can be introduced into the inner liner in different ways. As already described, the anchoring recesses can be introduced directly during the production of the interior lining.

A preferred embodiment of the method is characterized in that the creation of the anchoring recesses in method step b) comprises drilling, punching, cutting, or, preferably, a laser processing method, preferably by means of laser ablation. Due to the speed, simplicity, and flexibility, creating the anchoring depressions by laser ablation is preferred. In laser processing methods, it can be advantageous to introduce into the inner lumen a mandrel or another object, preferably made of metal, which absorbs the laser beam at the latest after having penetrated the wall of the inner liner, since otherwise a further, under certain circumstances undesirable, anchoring depression could be created in the opposite wall by the laser beam “shooting through.” While this can be desirable in some embodiments, it could create anchoring recesses in positions, e.g., under or even through the already arranged flexible circuit board, which are disadvantageous. For example, an electrical conductor on the flexible circuit board could be accidentally damaged in the process.

A preferred embodiment of the method is characterized in that the sheathing, in particular the introduction of the outer insulation into the anchoring recesses, in method step e) comprises heating the outer insulation. Although the sheathing can be carried out solely by applying pressure to the outer insulation and inner liner, depending upon the properties of the employed materials, it simplifies the method, however, and minimizes the risk of at least partial destruction of the individual components should the outer insulation be softened by heating. Heating can be carried out as part of extrusion of the outer insulation around the inner liner or as part of exertion of pressure on the outer insulation - for example, using heat-shrink tube.

The electrical contact with the at least one electrode in method step g) can be realized in different ways.

A preferred embodiment of the method is characterized in that, for establishing electrical contact with the electrode, preferably for all electrodes, at least one contact opening is created in the outer insulation in method step g) in order to selectively establish electrical contact between the electrode and at least one electrical conductor, accessible via the contact opening, of the flexible circuit board.

To create the contact opening, part of the outer insulation and, if necessary, part of the insulating layer of the flexible circuit board is removed at the corresponding position so that the at least one electrical conductor is exposed radially from the outside via the contact opening to be electrically contactable. The removal of parts of the insulating layer of the flexible circuit board is in particular necessary if the flexible circuit board does not already comprise an accessible electrical conductor at the position of the contact opening or has a contact surface via which the electrical conductor can be electrically contacted from the outside without removing parts of the insulating layer. At the position of the contact opening, electrical contact can be selectively, or in other words specifically, established between an electrode and the electrical conductor over the course of the subsequent method. If the catheter body is to have more than one electrode, in particular at the distal catheter body end, it is preferred that a separate contact opening be created for each of these electrodes, wherein electrical contact with one of the electrical conductors of the flexible circuit board is selectively established via each of these contact openings. For this purpose, the flexible circuit board preferably has at least exactly as many electrical conductors as electrodes that are to be attached to the distal catheter body end.

The contact opening can be created in different ways. For example, the contact opening can be created by cutting, punching, or by means of a laser processing method, preferably by laser ablation.

As already mentioned, the arrangement of the flexible circuit board in method step d) can be carried out in different ways - for example, also using an at least temporary fastening of the flexible circuit board on the inner liner outer surface. The attachment can be achieved with an adhesive such as glue or adhesive tape. Furthermore, the flexible circuit board can be oriented differently on the inner liner outer surface. For example, the flexible circuit board can be arranged substantially along the longitudinal axis of the inner liner. In this embodiment, the longitudinal axes of the flexible circuit board and the inner liner are aligned substantially parallel to each other. This is a particularly simple type of arrangement.

A preferred embodiment of the method is characterized in that the arrangement in method step d) comprises a spiral winding of the flexible circuit board in windings around the inner liner, and therefore onto the inner liner outer surface. In this embodiment, the flexible circuit board is therefore arranged axially in spiral windings, preferably along substantially the entire length of the inner liner.

The spiral arrangement of the flexible circuit board can provide the finished catheter body a particular resistance to kinking or breaking the electrical conductors of the flexible circuit board, since the windings can allow for improved bending of the same. Furthermore, the arrangement in windings increases the flexibility of establishing the electrical contact of the electrodes of the catheter body, which therefore does not always have to take place on the same side of the inner liner. Particularly with electrodes that are not ring electrodes and therefore do not extend around the entire circumference of the catheter body, the flexibility of the method with regard to the positioning of the electrodes is increased.

The number of windings and the distance between two adjacent windings can depend upon various factors, such as the width of the flexible circuit board and the number of electrical conductors.

Furthermore, in this preferred embodiment, the anchoring recesses are created in such a way that, in a plan view of the catheter body perpendicular to its longitudinal axis, i.e., in a side view, at least one anchoring recess is positioned between each two axially adjacent windings. Due to the spiral winding of the flexible circuit board, a spiral-shaped gap between the windings results. This gap has anchoring recesses, viz., in such a way that, in the described side view which shows an observer approximately half of the total circumference of the catheter body, at least one anchoring recess is or was created between each two axially adjacent windings. If one maintains the viewing angle and rotates the catheter body around its longitudinal axis so that the section of the catheter body facing the observer changes with the rotation, at least one anchoring depression can always be seen between two axially adjacent windings. This embodiment ensures that sufficient anchoring recesses are created between the windings and are at least partially filled with the outer insulation, so that the flexible circuit board is essentially firmly fixed between the inner liner and the outer insulation. This can simplify the subsequent method steps, in particular the installation of the electrodes, and reduce the risk of damage to the flexible circuit board, in particular to its electrical conductors, when the catheter body is bent.

The inner liner can comprise different thermosets or thermoset mixtures - preferably consist of different thermosets or thermoset mixtures.

A preferred embodiment of the method is characterized in that the inner liner comprises a polyimide (PI) or a polyether ether ketone (PEEK). These polymers are easily accessible, have good processability, and are also available economically.

### BRIEF DESCRIPTION OF THE DRAWINGS

The invention is further illustrated by way of example below by means of figures. The invention is not limited to the figures.

### In the figures:

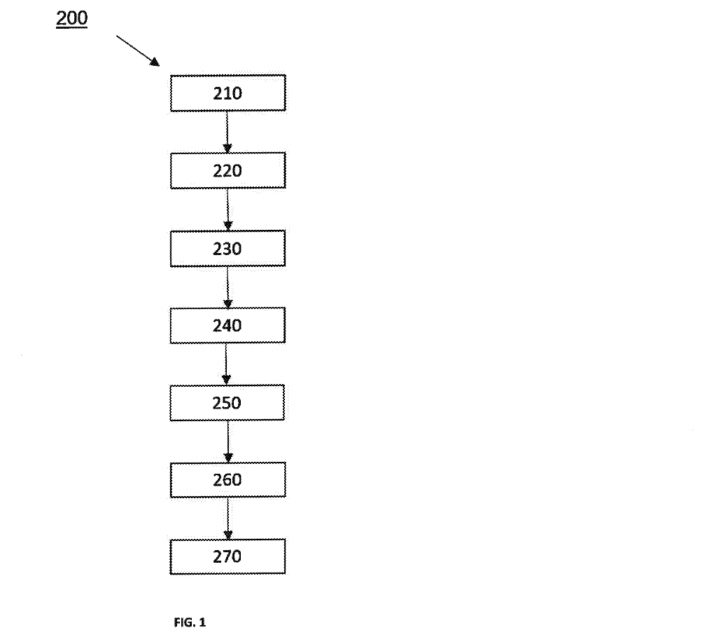

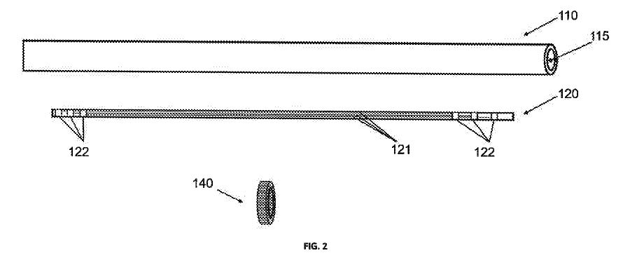

FIG. 1 shows an exemplary flowchart of a method for producing a catheter body;

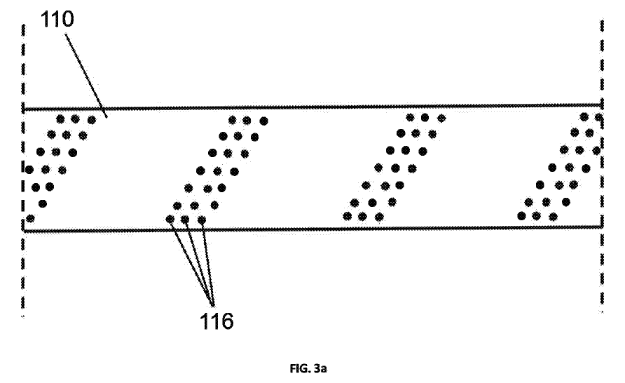

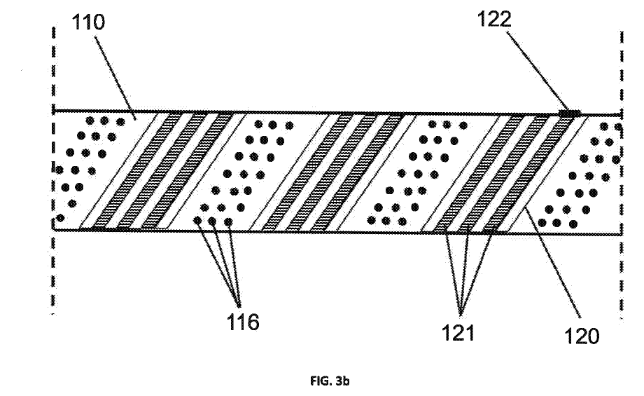

FIG. 2 shows exemplary schematic components for use in a method for producing a catheter body, in a perspectival side view;

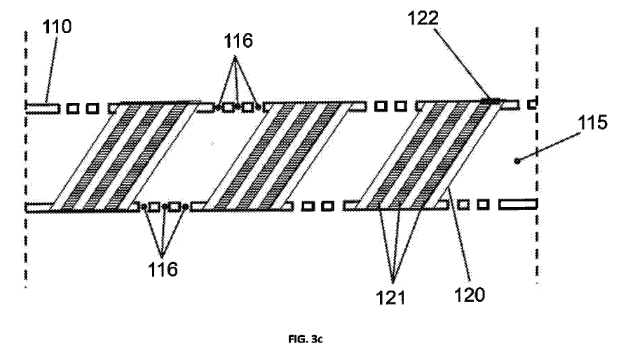

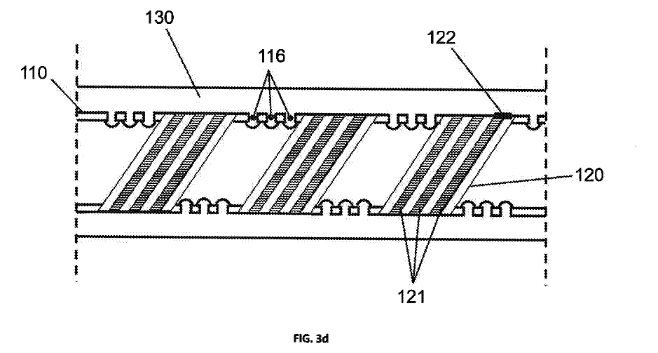

FIGS. 3a-e show exemplary method steps or intermediate products 3a to 3e of the method from FIG. 1 with the components from FIG. 2; and,

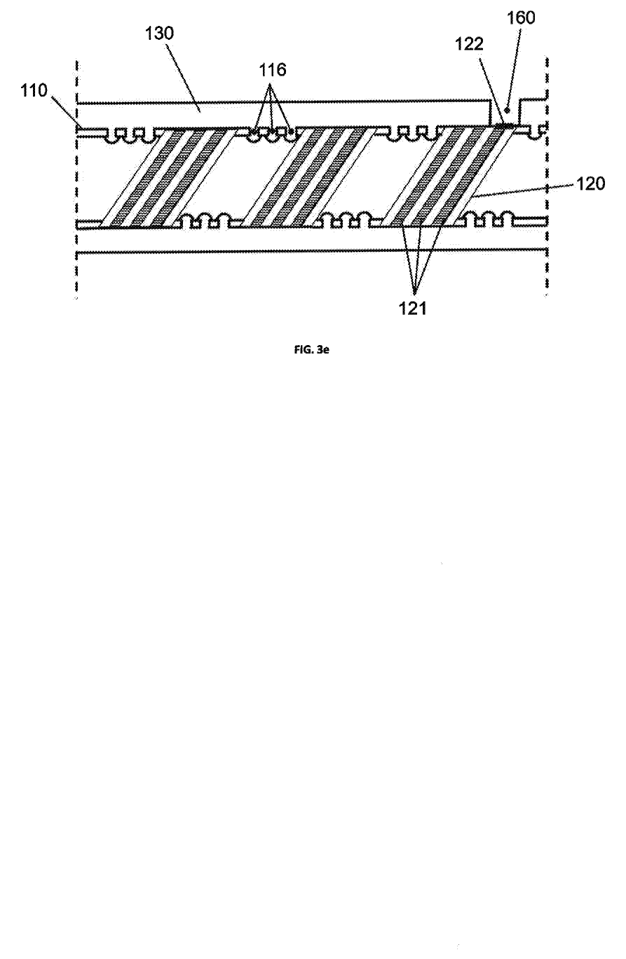

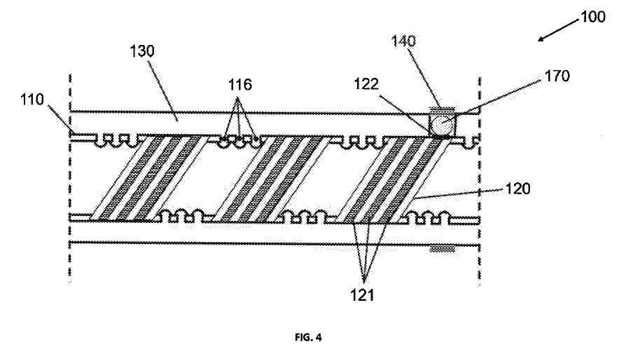

FIG. 4 shows an exemplary catheter body in a schematic longitudinal section.

### DETAILED DESCRIPTION OF THE INVENTION

FIG. 1 shows a flowchart of an exemplary method 200 for producing a catheter 100 comprising the method steps 210 to 270.

In a method step 210, a hollow-cylindrical inner liner 110 with an inner lumen 115 and a radially outer inner liner outer surface is provided. The inner liner 110 comprises a thermoset.

In a method step 220, a plurality of anchoring recesses 116 are created in the inner liner 110 which extend from the inner liner outer surface in the direction of the inner lumen 115. Preferably, the production is carried out by means of laser ablation, in such a way that the anchoring recesses 116 extend from the inner liner outer surface into the inner lumen 115 of the inner liner 110, i.e., radially through the entire inner liner 110.

In a method step 230, a flexible circuit board 120 comprising a plurality of electrical conductors 121 is provided. The electrical conductors 121 serve to electrically connect electrodes 140, preferably at a distal end of the catheter body 100, to further electrodes, often also referred to as connectors, or other devices and systems at a proximal end, opposite the distal end, of the catheter body 100. The flexible circuit board 120, or its electrical conductor 121, therefore extends over large parts, e.g., over at least 70%, of a total length of the final catheter body 100.

In a method step 240, the flexible circuit board 120 is arranged on the inner liner 110, in particular, the inner liner outer surface. The arrangement can be carried out in different ways, wherein it is preferred that the flexible circuit board 120 be wound spirally in windings around the inner liner 110. The arrangement can be supported, at least temporarily, by an adhesive

for example, an adhesive or an adhesive tape, in particular a double-sided adhesive tape

The arrangement of the flexible circuit board 120 or the creation of the anchoring recesses 116 in method step 220 is preferably carried out in such a way that, in a lateral view perpendicular to the longitudinal axis of the inner liner 110, at least one anchoring recess 116 is positioned between each two axially adjacent windings of the flexible circuit board 120.

In a method step 250, the inner liner 110 and the flexible circuit board 120 arranged on the inner liner 110 are sheathed with an outer insulation 130 comprising a thermoplastic, preferably consisting of a thermoplastic. This can be done, for example, by pulling a hollow-cylindrical outer insulation 130 onto the inner liner 110 and the flexible circuit board 120 arranged on the inner liner 110. Preferably, the sheathing is carried out by extrusion of, in particular coaxial extrusion of, or in other words “extrusion on,” a thermoplastic or a thermoplastic mixture around the inner liner 110 and the flexible circuit board 120. The sheathing further comprises at least partially inserting the outer insulation 130 into the anchoring recesses 116 so that the adhesion between the inner liner 110 and the outer insulation 130 is improved. Preferably, the flexible circuit board 120 is fixed between the inner liner 110 and the outer insulation 130 so that the position of the flexible circuit board 120 is basically unchangeable, and therefore the implementation of the further method steps of method 200 is simplified.

In a method step 260, at least one electrode 140 is provided. Preferably, the electrode 140 comprises a metal. More preferably, the electrode 140 is a ring electrode, preferably a metallic ring electrode.

In a method step 270, electrical contact is established between the at least one electrode 140 and at least one electrical conductor 121, preferably just one electrical conductor 121 of the flexible circuit board 120. Preferably, establishing electrical contact comprises creating at least one contact opening 160 in the vicinity of a distal catheter body end. The contact opening 160 serves to expose the at least one electrical conductor 121 of the flexible circuit board 120 so that it can be electrically contacted from outside the catheter body 100. To do this, at least parts of the outer insulation 130 of the electrical conductor 121 are removed above the position to be contacted. If the electrical conductor 121 is covered by one or more insulating layers of the flexible circuit board 120 at this position, the creation of the contact opening 160 also includes the removal of this/these insulating layer(s). The contact opening 160 is preferably created by laser ablation. Preferably, the electrical conductor 121 at the corresponding position comprises a contact surface 122, which can be freely electrically contacted from outside the flexible circuit board, such that (partial) removal of insulating layers of the flexible circuit board 120 is not necessary. Establishing the electrical contact can also include the use of an electrically conductive bridge element 170 such as a solder or a conductive adhesive which is applied between the electrical conductor 121 and the electrode 140.

The method steps 210 to 270 can be carried out in any order to the extent technically possible and reasonable. Preferably, the method steps 210 to 270 are carried out in the ascending numerical sequence described here.

FIG. 2 schematically shows various exemplary components of the method 200 for producing a catheter body 100 in a perspectival side view. Shown is a hollow-cylindrical inner liner 110 made of a polyimide with an inner lumen 115 that extends axially through the inner liner; an electrode 140 in the form of a metallic ring electrode; as well as a flexible circuit board 120. The flexible circuit board 120 comprises three electrical conductors 121, each extending from one axial end of the flexible circuit board 120 to an opposite axial end of the flexible circuit board 120. At both ends of the flexible circuit board 120, the electrical conductors 121 selectively terminate in each case in three contact surfaces 122 which are designed to selectively establish electrical contact with one of the electrical conductors 121 at the distal end and/or at the proximal end of the flexible circuit board 120. In other words, each of the electrical conductors 121 extends from one of the contact surfaces 122 at one end of the flexible conductor 120 to one of the contact surfaces 122 at the opposite end of the flexible conductor 120. In the generally preferred embodiment of the flexible circuit board 120, the electrical conductors 121, up to the contact surfaces 122 at both ends thereof, are electrically insulated from the outside, preferably by embedding in one or more electrically insulating layers. The contact surfaces 122, on the other hand, can be electrically contacted from the outside and are therefore freely accessible.

FIGS. 3a-e show various intermediate products or method steps of the method 200 from FIG. 2 based upon the components of FIG. 2.

FIG. 3a shows a section of the inner liner 110 in a plan view perpendicular to the longitudinal axis of the inner liner 110, where a plurality of helically surrounding anchoring recesses 116 (provided with reference signs only as an example) were created in the inner liner 110 by means of laser ablation (the rearward-extending anchoring recesses 116 as well as the anchoring recesses 116 in the plane of the drawing are not visible in the plan view). The anchoring recesses 116 extend from a radially outer inner liner outer surface of the inner liner 110 into the inner lumen 115 (see, for example, FIG. 2) of the inner liner 110 (see, for example, FIG. 3c). The anchoring recesses 116 therefore extend completely through the wall of the inner liner 110. In the shown embodiment, the anchoring recesses 116 have a substantially round cross-section. In further embodiments (not shown), the anchoring recesses 116 can, for example, have an oval, angular, e.g., square or hexagonal, cross-section.

FIG. 3b shows the section of the inner liner 110 from FIG. 3a, wherein the flexible circuit board 120 has been spirally wound in windings around the inner liner 110 so that the flexible circuit board 120 is arranged on the inner liner 110. The flexible circuit board 120 has been arranged in such a way that none of the anchoring recesses 110 are covered by the flexible circuit board 120. Furthermore, in the shown plan view, which shows the inner liner 110 perpendicular to its longitudinal axis and therefore also perpendicular to the longitudinal axis of the final catheter body 100 (see FIG. 4), it can be seen that a plurality of anchoring recesses 116 are positioned between each two axially adjacent windings of the flexible circuit board 120. In this way, in the further course of the method 200, the flexible circuit board 120 can be fixed between the inner liner 110 and the outer insulation 130 (see, for example, FIG. 3d), so that the circuit board's position and orientation basically do not change, even when the catheter body 100 is bent. One of the electrical conductors 121 has one of its contact surfaces 122 in the section shown.

FIG. 3c shows the same section as FIG. 3b, with the inner liner 110 shown in a schematic longitudinal section. For better clarity, the flexible circuit board 120 is also shown in a top view. In FIG. 3c, it can be seen that the anchoring recesses 116 (provided with reference signs only by way of example) extend completely from the inner liner outer surface into the inner lumen 115 of the inner liner 110.

FIG. 3d shows the section from FIGS. 3b and 3c, wherein the inner liner 110 and the flexible circuit board 120 have been sheathed with an outer insulation 130 made of a thermoplastic. In the shown embodiment, the sheathing 250 was carried out by means of coaxial extrusion of a thermoplastic. At the same time, the outer insulation 130 has been introduced into the anchoring recesses 116. In the shown embodiment, the outer insulation 130 has been introduced into the inner lumen 115 of the inner liner 110 so that a kind of mushroom-shaped undercut made of the thermoplastic has been formed there (shown exaggerated), which further improves the adhesion between the outer insulation 130 and the inner liner 110. The undercut made of the thermoplastic makes it even more difficult for the outer insulation 130 to detach from the inner liner 110. The flexible circuit board 130 (further shown in a plan view) is therefore arranged and substantially fixed radially between the inner liner 110 and the outer insulation 130, also due to the positioning of the anchoring recesses 116.

FIG. 3e shows the section from FIG. 3d, wherein a contact opening 160 has been created in the outer insulation 130, through which one of the electrical conductors 121 of the flexible circuit board 120 can be electrically contacted via its contact surface 122 accessible from the outside via the contact opening 160. In the shown embodiment, the contact surface 160 was created by laser ablation.

FIG. 4 shows a section of the catheter body 100 produced by way of example from the intermediate product from FIG. 3e, wherein, for this purpose, a bridge element 170 in the form of a conductive adhesive, e.g., comprising a silver powder dispersed in a curable organic medium, has been introduced into the contact opening 160 (see FIG. 3e). After the bridge element 170 was inserted, the ring electrode 140 was pushed onto the arrangement at the axial height of the contact opening 160 and in contact with the bridge element 170. By curing the bridge element 170, an electrical contact is made between the ring electrode and the corresponding electrical conductor 121 - in the shown embodiment, indirectly via the bridge element 170.

### REFERENCE SIGNS

100 Catheter body

110 Inner liner

115 Inner lumen

116 Anchoring recess

120 Flexible circuit board

121 Electrical conductor

122 Contact surface

130 Outer insulation

140 Electrode

160 Contact opening

170 Bridge element

200 Method for producing a catheter body

210 Providing an inner liner

220 Creating anchoring recesses

230 Providing a flexible circuit board

240 Arranging the flexible circuit board

250 Sheathing the inner liner and the flexible circuit board with an outer insulation

260 Providing an electrode

270 Establishing electrical contact with the electrode

What is claimed is:

1. A method for producing a catheter body, comprising the method steps of:
a) providing a hollow-cylindrical inner liner having an inner lumen and a radially outer inner liner outer surface, wherein the inner liner comprises a thermoset;
b) creating a plurality of anchoring recesses in the inner liner which extend from the inner liner outer surface toward the inner lumen;
c) providing a flexible circuit board comprising a plurality of electrical conductors;
d) arranging the flexible circuit board on the inner liner outer surface;
e) sheathing the inner liner and the flexible circuit board with an outer insulation, wherein the outer insulation comprises a thermoplastic and is at least partially introduced into the anchoring recesses;
f) providing at least one electrode; and,
g) establishing electrical contact between the at least one electrode and at least one of the electrical conductors of the flexible circuit board.

2. The method according to claim 1, wherein the anchoring recesses have a diameter in a range between 5 µm and 200 µm.

3. The method according to claim 2, wherein the anchoring recesses have a radial depth which is greater than their diameter.

4. The method according to claim 1, wherein the anchoring recesses extend from the inner liner outer surface into the inner lumen.

5. The method according to claim 4, wherein in method step e), the outer insulation is introduced via the anchoring recesses into the inner lumen.

6. The method according to claim 1, wherein the sheathing in method step e) comprises extruding the outer insulation.

7. The method according to claim 1, wherein the sheathing in method step e) comprises exerting pressure on the outer insulation in the direction of the inner lumen.

8. The method according to claim 7, wherein the pressure is exerted on the outer insulation by means of a heat-shrink tube.

9. The method according to claim 7, wherein the outer insulation is formed by heating a heat-shrink tube.

10. The method according to claim 1, wherein the production of the anchoring recesses in method step b) comprises drilling, punching, cutting, or a laser processing method.

11. The method according to claim 1, wherein the sheathing in method step e) comprises heating the outer insulation.

12. The method according to claim 1, wherein the inner lining provided in method step a) has a wall thickness in a range between 10 µm and 200 µm.

13. The method according to claim 1, wherein for establishing electrical contact with the electrode in method step g), at least one contact opening is created in the outer insulation in order to selectively establish electrical contact between the electrode and at least one electrical conductor, accessible via the contact opening, of the flexible circuit board.

14. The method according to claim 1, wherein the arrangement in method step d) comprises spiral winding of the flexible circuit board in windings around the inner liner, and wherein the creation of the anchoring recesses in method step b) takes place in such a way that, in a plan view of the catheter body, perpendicular to its longitudinal axis, at least one anchoring recess is positioned between every two axially adjacent windings.

15. The method according to claim 1, wherein the inner liner comprises a polyimide or a polyether ether ketone.

16. The method according to claim 2, wherein the anchoring recesses extend from the inner liner outer surface into the inner lumen.

17. The method according to claim 3, wherein the anchoring recesses extend from the inner liner outer surface into the inner lumen.

18. The method according to claim 2, wherein the sheathing in method step e) comprises extruding the outer insulation.

19. The method according to claim 3, wherein the sheathing in method step e) comprises extruding the outer insulation.

20. The method according to claim 4, wherein the sheathing in method step e) comprises extruding the outer insulation.
